(19) United States
(12) Patent Application Publication
(10) Pub. No.: US 2024/0166577 A1
(43) Pub. Date: May 23, 2024

(54) ADIABATICALLY CONDUCTED PROCESS FOR THE PRODUCTION OF 1,3-BUTADIENE FROM MIXTURES OF ETHANOL AND ACETALDEHYDE WITH CATALYST REGENERATION

(71) Applicant: SYNTHOS DWORY 7 SPOLKA Z OGRANICZONA ODPOWIEDZIALNOSCIA, OSWIECIM (PL)

(72) Inventors: Ewelina JANKOWIAK, OSWIECIM (PL); Szymon SKOWRONEK, OSWIECIM (PL); Magdalena SITKO, OSWIECIM (PL); Joanna KRYCA, OSWIECIM (PL)

(21) Appl. No.: 18/552,779

(22) PCT Filed: Mar. 31, 2022
(86) PCT No.: PCT/EP2022/058716

§ 371 Date: Sep. 27, 2023

(30) Foreign Application Priority Data
Apr. 1, 2021 (EP) 21461530.4

(51) Int. Cl. C07C 1/207; B01J 8/02; B01J 23/20; B01J 23/92; B01J 38/20; C07C 45/29

(52) U.S. Cl. CPC C07C 1/2076; B01J 8/0278; B01J 8/0285; B01J 23/20; B01J 23/92; B01J 38/20; C07C 45/29; B01J 2208/00176; B01J 2208/00752; C07C 2523/20

(57) ABSTRACT

The invention relates to a process for the production of 1,3-butadiene from ethanol and acetaldehyde with catalyst regeneration comprising a) reacting a feed comprising ethanol and acetaldehyde in a reactor having at least one adiabatic reaction zone comprising a supported catalyst, and b) regenerating the supported catalyst. Regeneration stage b) comprises stripping step i. at a temperature of 300 to 400° C., ii. first and second combustion steps ii. and iii. at a temperature of 350 to 400° ° C. and 400 to 550° C. respectively, and stripping step iv. at a temperature of 550° C. to 300° ° C. The gas flows to each of regeneration steps b)i. to b)iv. are first heated and then contact the supported catalyst.

The present invention relates to a process for the production of 1,3-butadiene from ethanol and acetaldehyde with catalyst regeneration, the process comprising a) reacting a feed comprising ethanol and acetaldehyde in a reactor having at least one adiabatic reaction zone, the adiabatic reaction zone comprising a supported catalyst and producing 1,3-butadiene, whereby spent supported catalyst is formed; and b) regenerating the spent supported catalyst in a regeneration stage. Moreover, the invention relates to a process for the production of 1,3-butadiene from ethanol with catalyst regeneration. The invention further relates to a plant for the production of 1,3-butadiene comprising at least one reactor, the reactor having at least one zone for producing 1,3-butadiene, the zone comprising a supported catalyst, the reactor having reactant heating means sufficient to react ethanol and acetaldehyde in the zone under adiabatic conditions, the reactor further having c) means for regenerating the supported catalyst. Also, the invention relates to a plant for the production of 1,3-butadiene from ethanol.

### BACKGROUND

1,3-Butadiene is one of the key chemicals in the polymer industry and is mainly used to manufacture synthetic rubbers. In a classic approach, 1,3-butadiene is produced on industrial scale via steam cracking of naphtha, and is separated from the effluent by extractive distillation. However, major disadvantages of this process are a high energy consumption and the reliance on fossil fuel feedstock. The risk of fossil fuel depletion as well as the increasing requirements for environmental protection drive the search for lower energy-consuming and more environmentally-benign routes for olefins production, preferably based on renewable resources, such as biomass.

Economic and environmental considerations have led to ethanol as one of the most promising sustainable feedstocks for 1,3-butadiene production. Two commercially interesting routes for the chemical conversion of ethanol to 1,3-butadiene conversion are known: The so-called one step (Lebedev) process and the so-called two step (Ostromislensky) process. The one step process includes the direct catalytic conversion of gaseous ethanol to 1,3-butadiene. The two step process divides the reaction into two stages—i) partial dehydrogenation of ethanol to acetaldehyde and ii) conversion of a mixture of ethanol and acetaldehyde to 1,3-butadiene.

The conversion of a mixture of ethanol and acetaldehyde to 1,3-butadiene is an endothermic reaction. Maintaining a temperature in the reactor that delivers sufficient energy for the optimal conversion of the substrates to 1,3-butadiene is essential. Thus, carrying out the conversion of a mixture of ethanol and acetaldehyde to 1,3-butadiene via isothermal processes over dedicated catalysts is well known in the literature and many modifications of isothermal processes have been reported. When the conversion of a mixture of ethanol and acetaldehyde to 1,3-butadiene is carried out under isothermal conditions, the reactor and catalyst therein are heated by means of a heat transfer medium, to maintain a relatively constant temperature that is high enough to allow for the endothermic conversion of ethanol/acetaldehyde mixtures to 1,3-butadiene to take place. However, the use of heat transfer media, so as to provide the high reaction temperatures required especially for regeneration, such as molten salt fluids, is expensive and makes the reactor set-up more complicated. Regeneration at a temperature of up to 550° C. is typically required to refresh (rejuvenate) the catalyst for the production of 1,3-butadiene, under (at least in part) oxidative conditions, whereas the reaction of ethanol with acetaldehyde to 1,3-butadiene is typically performed at a temperature of 320 to 420° C., such as about 350° C. Also, reactor maintenance is more difficult when employing the typical equipment used for isothermal processes, due to the presence of the heat transfer devices. Moreover, isothermal reactors are often complicated in terms of construction as they are often multi-tubular reactors. This is particularly laborious because the life-time of typical catalysts, such as tantalum catalysts, for the production of 1,3-butadiene is relatively short, and the catalyst loading needs to be changed regularly, e.g. after about 1 to 2 years.

WO2020/126921 A1 teaches a process for the production of butadiene from ethanol with catalyst regeneration. Typically, there are three reactors (two in the catalytic reaction stage and one in the regeneration stage). WO2020/126920 A1 also teaches a process for the production of butadiene from ethanol with catalyst regeneration and, typically, there are six reactors (four in the catalytic reaction stage and two in the regeneration stage). Similar to the teaching of WO2020/126921 A1, the sequence of reaction and regeneration steps of WO2020/126920 A1 is governed by the need to specifically perform regeneration for half the catalytic cycle time.

WO 2020/126920 A1 and WO 2020/126921 A1 teach a four-phase regeneration. After a first (stripping) phase, at a temperature between 300 and 400° ° C. under a flow of inert gas, there are a first combustion phase under a gas flow comprising inert gas and oxygen at a content of 1 vol. % or less at a temperature between 300 and 450° C., a second combustion phase under a gas flow comprising inert gas and oxygen at a content of 2 vol. % or greater and at a temperature between 390 and 550° C., and a final stripping phase under a flow of inert gas at a temperature between 550 and 300° C. However, the reaction/regeneration sequence according to WO 2020/126920 A1 and WO 2020/126921 A1 is undesired because it strictly requires the reactors to be in regeneration mode for half the time that the reactors are in catalytic reaction mode.

CN 101927180 B relates to zeolite catalysts for preparing propylene from C4 olefins, and the application and regeneration thereof. The use of the catalysts in a fixed bed adiabatic reactor to generate a reactant mixture containing ethylene and propylene is described. The acidity of the zeolite catalysts disclosed in CN 101927180 B makes them suitable for the cracking of C4 olefins into propylene and ethylene.

Hence, there is a need for providing a process for the production of 1,3-butadiene from mixtures of ethanol and acetaldehyde, including regeneration, that is more economical and allows a more simple reactor set-up and maintenance.

### SUMMARY OF THE INVENTION

According to the present invention, it was surprisingly found that a process for the production of 1,3-butadiene from ethanol and acetaldehyde with catalyst regeneration can be conducted in a much more economical manner and allows for a simpler reactor set-up and maintenance, provided the reaction stage is conducted under adiabatic conditions and catalyst regeneration is carried out in four specific steps.

Due to the endothermic nature of the conversion of the mixture of ethanol and acetaldehyde to 1,3-butadiene, an effective, uniform, and easily-controllable supply of heat to the reaction zone is one of the key factors for proper reactor design. To meet these requirements, the reactor must be characterized by a high ratio of heat transfer area to reaction volume. Thus, a typical reactor design for such application is a multitube fixed-bed reactor of the shell-and-tube heat exchanger type, where a heating medium flows through the shell and the reactants flow through the small diameter tubes (loaded with catalyst grains). Such multitube reactor is very challenging to design, in particular when a heating medium is required that is suitable for the high temperature needed for regenerating the (supported) catalyst. Furthermore, such kind of reactor is challenging to operate and to maintain, in particular when having to replace used catalyst with new catalyst. Because the conversion of the mixture of ethanol and acetaldehyde to 1,3-butadiene is carried out according to the present invention in an adiabatic thermal mode, heat supply is considered separately from reactor design, and the reactor and catalytic zone therein can be shorter and have a larger diameter. For instance, reactors of the adiabatic tubular fixed-bed type, as used in accordance with the invention, provide a simple design, are of straightforward construction and allow easy operation and maintenance.

Therefore, in a first aspect, the present invention relates to a process for the production of 1,3-butadiene from ethanol and acetaldehyde with catalyst regeneration, the process comprising
a) reacting a feed comprising ethanol and acetaldehyde in a reactor having at least one adiabatic reaction zone, the adiabatic reaction zone comprising a supported catalyst and producing 1,3-butadiene, whereby spent supported catalyst is formed;
b) regenerating the spent supported catalyst in a regeneration stage comprising four subsequent steps.

In a second aspect, the present invention relates to a process for the production of 1,3-butadiene from ethanol with catalyst regeneration comprising
x) producing acetaldehyde from ethanol in an acetaldehyde producing reactor having a reaction zone, the reaction zone of the acetaldehyde producing reactor comprising a supported or unsupported (bulk) catalyst, and
y) producing 1,3-butadiene from ethanol and acetaldehyde with catalyst regeneration according to the process of the first aspect of the invention.

In a third aspect, the present invention relates to a plant for the production of 1,3-butadiene comprising at least one reactor for producing 1,3-butadiene from ethanol and acetaldehyde, the reactor for producing 1,3-butadiene having
a) at least one zone for producing 1,3-butadiene, the zone comprising a supported catalyst for producing 1,3-butadiene, and
b) means for feeding a feed comprising ethanol and acetaldehyde into the reactor for producing 1,3-butadiene,
the reactor for producing 1,3-butadiene having reactant heating means for heating the feed comprising ethanol and acetaldehyde before contacting the supported catalyst for producing 1,3-butadiene, the reactant heating means being sufficient to react ethanol and acetaldehyde under adiabatic conditions,
the reactor for producing 1,3-butadiene further having
c) means for regenerating the supported catalyst for producing 1,3-butadiene, comprising
   x) means for feeding a flow comprising inert gas into the reactor for producing 1,3-butadiene, and
   y) means for feeding a flow comprising oxygen into the reactor for producing 1,3-butadiene,
the reactor for producing 1,3-butadiene having regenerant heating means for heating a flow comprising the inert gas and the oxygen before contacting the supported catalyst for producing 1,3-butadiene, the regenerant heating means being sufficient to regenerate the supported catalyst under adiabatic conditions.

In a fourth aspect, the present invention relates to a plant for the production of 1,3-butadiene from ethanol, having
i. at least one reactor for producing acetaldehyde from ethanol, the reactor for producing acetaldehyde having
   a) at least one zone for producing acetaldehyde from ethanol, the zone for producing acetaldehyde comprising a supported or unsupported (bulk) catalyst for producing acetaldehyde, and
   b) means for feeding a feed comprising ethanol into the reactor for producing acetaldehyde; and
ii. at least one reactor according to the third aspect.

### DETAILED DESCRIPTION OF THE INVENTION

Process for the Production of 1,3-butadiene from Ethanol and Acetaldehyde with Catalyst Regeneration

According to the first aspect of the invention, the process for the production of 1,3-butadiene from ethanol and acetaldehyde with catalyst regeneration comprises
a) reacting a feed comprising ethanol and acetaldehyde in a reactor having at least one adiabatic reaction zone, the adiabatic reaction zone comprising a supported catalyst and producing 1,3-butadiene, whereby spent supported catalyst is formed;
b) regenerating the spent supported catalyst in a regeneration stage comprising the following subsequent steps:
   i. a stripping step, carried out at a temperature in a range of from 300 to 400° C., by contacting the supported catalyst with a gas flow comprising inert gas, the gas flow having an oxygen content of 200 vol.-ppm or less;
   ii. a first combustion step carried out at a temperature in a range of from 350 to 400° C., by contacting the supported catalyst with a gas flow comprising inert gas, the gas flow having an oxygen content in a range of from 0.2 to 8 vol. %;
   iii. a second combustion step carried out at a temperature in a range of from 400 to 550° C., by contacting the supported catalyst with a gas flow comprising inert gas, the gas flow having an oxygen content in a range of from 0.2 to 8 vol. %;
   iv. a stripping step carried out at a temperature in a range of from 550° C. to 300° C., by contacting the supported catalyst with a gas flow comprising inert gas, the gas flow having an oxygen content of less than 200 vol.-ppm;
   wherein the gas flows to each of regeneration steps b)i. to b)iv. are first heated and then contact the supported catalyst.

According to the invention, a heat exchanger train is designed specifically to supply heat to the feed that acts as heat carrier, and the reactor design focuses on a diminution of heat losses. In reaction stage a), the feed comprising ethanol and acetaldehyde acts as heat carrier for effecting the endothermic reaction to 1,3-butadiene under adiabatic conditions. In regeneration stage b), the respective heated gas flows act as heat carrier for regenerating the supported catalyst under adiabatic conditions.

Reaction Stage a)

According to a first aspect of the invention, the process for the production of 1,3-butadiene comprises reacting a feed comprising ethanol and acetaldehyde in a 1,3-butadiene producing reactor having at least one adiabatic reaction zone, the adiabatic reaction zone comprising a supported catalyst and producing 1,3-butadiene.

According to the present invention, the heat energy required for the (endothermic) reaction of the mixture of ethanol and acetaldehyde, to give 1,3-butadiene, is supplied to the adiabatic reaction zone only by the feed supplied to the adiabatic reaction zone. Said feed supplied to the adiabatic reaction zone is consequently heated to a suitable temperature by heating means, before the contacting of the feed supplied to the adiabatic reaction zone with the supported catalyst takes place.

Preferably, all materials provided to the supported catalyst in reaction stage a) are first mixed and then heated, and are then provided to the reaction zone, i.e. a mixed feed comprising ethanol and acetaldehyde is heated, and then contacts the supported catalyst.

The heating means for increasing the temperature of the feed supplied to the adiabatic reaction zone may be, for example, a heat exchanger or a heated inert packing separating two adiabatic reaction zones within one reactor.

According to the invention, a heat exchanger train is designed specifically to supply heat to the feed that acts as heat carrier, and the reactor design focuses on a diminution of heat losses. In reaction stage a), the feed comprising ethanol and acetaldehyde acts as heat carrier for effecting the endothermic reaction to 1,3-butadiene under adiabatic conditions. In regeneration stage b), the respective heated gas flows act as heat carrier for regenerating the supported catalyst under adiabatic conditions.

The feed supplied to the adiabatic reaction zone comprises a feed comprising ethanol and acetaldehyde and, optionally, additional feed comprising acetaldehyde.

The effluent from the reaction zone, or, if several (n) reaction zones are used, the effluent from the n^{th} reaction zone, is separated and ethanol and acetaldehyde are purified to a certain purity level, before recycling them. Ethanol from the effluent may be recycled to the reaction zone producing acetaldehyde, or to the (first or any subsequent) reaction zone producing 1,3-butadiene, or to both the reaction zone producing acetaldehyde and the (first or any subsequent) reaction zone producing 1,3-butadiene. Acetaldehyde from the effluent may be recycled to the (first or any subsequent) reaction zone producing 1,3-butadiene.

Since, in the process according to the invention, the heat energy required for the endothermic reaction of the mixture of ethanol and acetaldehyde, to give 1,3-butadiene, is supplied to the adiabatic reaction zone by the feed supplied to the reaction zone, no additional heat supply to the adiabatic reaction zone is required. It was surprisingly found that even though the temperature decreases along the adiabatic reaction zone due to the endothermic effect of the reaction, the conversion of ethanol and acetaldehyde to 1,3-butadiene can be carried out efficiently without the provision of additional heating means for maintaining a constant temperature in the reaction zone, as long as the feed comprising ethanol and acetaldehyde has a suitably high temperature when entering the adiabatic reaction zone comprising a supported catalyst and producing 1,3-butadiene. The process according to the present invention is advantageous since it allows a much simpler and economical reactor set-up.

Preferably, the reaction zone or several reaction zones producing 1,3-butadiene and operating under adiabatic conditions are designed in terms of the temperature drop observed in each individual reaction zone such that each individual reaction zone operates within the temperature range providing good activity, conversion and selectivity towards 1,3-butadiene.

With the process according to the present invention as described herein, a mixture of ethanol and acetaldehyde can be converted to 1,3-butadiene with a conversion rate of about 35 to 45% and a selectivity to 1,3-butadiene of 70 to 75%.

Preferably, in the process according to the invention, the feed to the adiabatic reaction zone comprises at least 40 wt. %, more preferably at least 70 wt. %, of ethanol based on the total weight of the feed.

According to a preferred embodiment, the ethanol starting material used in the process according to the invention is aqueous ethanol, preferably is at least 80 wt. % aqueous ethanol, more preferably at least 90 wt. % aqueous ethanol, based on the total weight of the ethanol starting material.

According to another preferred embodiment, the ethanol starting material used in the process according to the invention comprises more than 90 wt. %, preferably more than 95 wt. %, more preferably more than 97 wt. %, most preferably more than 98 wt. % of ethanol, based on the total weight of the ethanol starting material.

Preferably, the feed comprises at least 12.5 wt. %, more preferably at least 20 wt. %, of acetaldehyde based on the total weight of the feed.

The acetaldehyde fed into the 1,3-butadiene producing reactor may be produced by an acetaldehyde producing reactor producing acetaldehyde from ethanol as described herein further below. Alternatively, the acetaldehyde may be obtained from the workup of the effluent from a reaction zone or reactor producing 1,3-butadiene.

In reaction stage a), the feed comprising ethanol and acetaldehyde is fed to the adiabatic reaction zone for a suitable time period, producing 1,3-butadiene. In a preferred embodiment, the total time period of reaction stage a) is less than 300 h, preferably in a range of from 50 to 250 h, more preferably in a range of from 100 to 200 h, in particular about 150 h.

With time, catalytic activity of the supported catalyst producing 1,3-butadiene falls to an undesirable level, and feeding of ethanol and acetaldehyde is discontinued.

In a preferred embodiment of the invention, and at the end of reaction stage a), a gas flow comprising inert gas (the gas flow having an oxygen content of less than 200 vol.-ppm) is fed into the reactor for producing 1,3 butadiene, and feeding of ethanol and acetaldehyde is stopped. This provides for inert keeping of the supported catalyst, immediately prior to regeneration stage b).

A preferred reactor design used according to the invention is a tubular fixed-bed reactor, which is advantageous because it is characterized by a small ratio of heat transfer area to reaction volume.

According to the present invention, the duration of catalytic stage a) is extended, as compared to the duration of regeneration stage b). Additionally, total lifetime of the supported catalyst is extended, which is a further advantage. Consequently, the invention allows for the implementing of a process for the production of 1,3-butadiene from ethanol and acetaldehyde with catalyst regeneration and replacement of supported catalyst, the process comprising the process according to the first aspect, and replacement of supported catalyst. Total lifetime of the supported catalyst until the supported catalyst has to be replaced, i.e. the length of all sequences of catalytic stage a), then regeneration stage b), followed by catalytic stage a) etc., is within a range of 6 months to 4 years, preferably 1 to 2 years.

During reaction stage a) according to the present invention, the supported catalyst gradually loses its activity after some time of operation, because substances causing deactivation of the active sites are adsorbed onto its surface. A common phenomenon is coking of the supported catalyst, which not only causes the speed of the stage a) reaction to 1,3-butadiene to drop noticeably. Most of all, coking increases the resistance to the flow of reactants through the bed, resulting in a pressure drop over the length of the reaction zone comprising the supported catalyst.

Moreover, total conversion of the reactants and selectivity to 1,3-butadiene, as well as productivity of the supported catalyst, decrease in reaction stage a) with time. Yield and productivity are related to both conversion and selectivity, thus they are relevant parameters to quantitatively identify spent supported catalyst. Regeneration is typically performed when yield is ca. 20% (conversion of ca. 30%, selectivity to 1,3-butadiene of ca. 68%).

In a preferred embodiment of the invention, at the end of reaction stage a), yield to 1,3-butadiene, as defined as (conversion×selectivity to 1,3-butadiene/100), is in a range of from 40 to 90%, preferably 50 to 85%, in particular from 60-80% of the newly-regenerated catalyst. Then, regeneration stage b) is started, and supported catalyst is regenerated.

In a preferred embodiment of the invention, at the end of reaction stage a), productivity to 1,3-butadiene, as defined as (amount of 1,3-butadiene produced per time unit), is in a range of from 40 to 90%, preferably 50 to 85%, in particular from 60-80% of the newly-regenerated catalyst. Then, regeneration stage b) is started, and supported catalyst is regenerated.

In a further preferred embodiment of the invention, regeneration of the supported catalyst is performed after each cycle of reaction stage a), in every battery of reactors connected in series. Typically, the carbon dioxide concentration in the reactor effluent is monitored during the entirety of catalytic regeneration stage b).

During regeneration stage b), the respective gas flows to steps i. to iv. (i.e. regenerants) provide for regeneration of the spent catalyst, so that, after regeneration stage b), further 1,3-butadiene can be produced in a follow-up reaction stage a). According to the present invention, the heat energy required is supplied to the supported catalyst by the respective heated gas flow (heated regenerant).

In another preferred embodiment of the invention, and immediately subsequent to reacting in stage a), in stripping step i. the heated gas flow comprising inert gas having a temperature in a range of from 300 to 400° C. contacts the supported catalyst, without any intermediate temperature reduction. Then, immediately subsequent to such stripping step i., in first combustion step ii. the heated gas flow comprising inert gas having a temperature in a range of from 350 to 400° C. contacts the supported catalyst.

In the process according to the first aspect of the present invention, regeneration stage b) is performed such that, in stripping step i., and first combustion step ii., carbonaceous deposits having a lower boiling point are removed from the supported catalyst by the gas flow. Additionally, and, in step ii., further carbonaceous deposits are removed from the supported catalyst by oxidation, at the moderate temperature in a range of from 350 to 400° C. That the gas flow in first combustion step ii. of the regeneration stage b) in the process of the invention may have an oxygen content in a range up to 8 vol. %, at a moderate temperature in a range of from 350 to 400° C., significantly shortens subsequent regeneration stages iii. and iv., thereby extending total lifetime of the supported catalyst, whilst at the same time allowing for a reduction of the total duration of regeneration stage b). It is an advantage of the present invention that the time period that the supported catalyst spends at a temperature of above 400° C., i.e. above the preferred temperature of reaction stage a), is substantially reduced. This is in contrast to the teaching of WO2020/126921 A1 and WO2020/126920 A1, which already in the first combustion stage (even though with a flow having a limited oxygen level of 1 vol. % or less) requires that the supported catalyst is exposed to a substantially higher temperature, of up to 450° C.

Also, and because the third regeneration step iii. (the second combustion step) is highly exothermic, the preceding stripping step i. and first combustion step ii. according to the present invention ensure that the supported catalyst has a reduced risk of being exposed to any temperature run-off of the supported catalyst during the third regeneration step iii., which run-off could otherwise reach or exceed 1000° C. Temperatures as high as 1000° C. or above would lead to catalyst sintering, and, consequently, to an undesired decrease in catalytic effectivity of the supported catalyst during any reaction stage a) subsequent to regeneration stage b). Consequently, stripping step i. and first combustion step ii., at a moderate temperature of up to 400° C., lead to a reduction of the time required for iii. second combustion step and iv. stripping step of the regeneration stage b) according to the process of the invention, whilst preserving catalyst lifetime. When performing regeneration stage b) in accordance with the present invention, local temperature maxima reaching (or even exceeding) 1000° C. are more easily avoided, even in second regeneration step ii.

Moreover, it is preferred that regeneration stage b) is performed, in stripping step i. and/or first combustion step ii., with a gas flow comprising, in addition to inert gas (and having the specified oxygen content), steam as a further component. Steam, which may be present in step i., in step ii., or in steps i. and ii., significantly decreases the risk of an exothermic run-off of the supported catalyst's temperature during combustion steps ii. and iii. Moreover, and in case there is a risk of a run-off of the temperature of the supported catalyst (i.e. local temperature maxima exceeding 400° C., when steam is present, or 1000° ° C., in the absence of steam), the process according to the present invention is very versatile because the first combustion step ii. is carried out at a moderate temperature, in a gas flow having an oxygen content in a range up to 8 vol. %, which reduces the risk of temperature run-off particularly in second combustion step iii. This allows for better control of regeneration stage b).

Preferably, the supported catalyst comprises one or more of tantalum, zirconium, niobium, hafnium, titanium, and tin. More preferably, the supported catalyst comprises tantalum. Most preferably, the supported catalyst comprises tantalum in an amount of from 0.1 to 10 wt %, such as from 0.5 to 5 wt %, e.g. from 2 to 3 wt %, calculated as Ta2O5 and based on the total weight of the supported catalyst.

Preferably, in reaction stage a), the 1,3-butadiene producing reactor includes a first adiabatic reaction zone comprising a supported catalyst and producing 1,3-butadiene and a second adiabatic reaction zone comprising a supported catalyst and producing 1,3-butadiene. More preferably, the first adiabatic reaction zone comprising a supported catalyst and producing 1,3-butadiene and the second adiabatic reaction zone comprising a supported catalyst and producing 1,3-butadiene are separated by a non-reaction zone. It is preferred that the non-reaction zone is heated. Most preferably, the heated non-reaction zone comprises an inert packing.

Preferably, in reaction stage a), an additional feed comprising acetaldehyde (and optionally ethanol) is fed into the reactor after the first adiabatic reaction zone comprising a supported catalyst and producing 1,3-butadiene. It is preferred that the additional feed is mixed with the effluent from the first adiabatic reaction zone comprising a supported catalyst and producing 1,3-butadiene and is then fed to the second adiabatic reaction zone comprising a supported catalyst and producing 1,3-butadiene.

Preferably, in reaction stage a), the additional feed further comprises ethanol, and the molar ratio of ethanol to acetaldehyde in the additional feed is in the range of from 0.1 to 5, preferably 1 to 2, more preferably 1.4 to 1.8.

It is preferred in all embodiments of the invention that, in reaction stage a), a first 1,3-butadiene producing reactor having at least a first adiabatic reaction zone comprising a supported catalyst and producing 1,3-butadiene, and a second 1,3-butadiene producing reactor having at least a second adiabatic reaction zone comprising a supported catalyst and producing 1,3-butadiene are connected in series, and at least part of the effluent (and preferably the entire effluent) from the first 1,3-butadiene producing reactor is fed to the second 1,3-butadiene producing reactor. Preferably, an additional feed comprising acetaldehyde (and optionally ethanol) is fed into the second reactor.

According to a preferred embodiment of the process according to the invention, the molar ratio of ethanol to acetaldehyde in the feed to the adiabatic reaction zone is in the range of from 1 to 7, preferably 1.5 to 5, more preferably 2 to 4, in particular 2.5 to 3.5, such as about 3.

Preferably, the weight hourly space velocity (WHSV) in the adiabatic reaction zone is in the range of from 0.5 to 10 h⁻¹, more preferably from 1.5 to 4 h⁻¹, most preferably from 2 to 3 h⁻¹.

Most preferably, the WHSV is adjusted such that the molar ratio of ethanol to acetaldehyde in the effluent from the adiabatic reaction zone is at least 20%, preferably at least 30%, higher than the molar ratio of ethanol to acetaldehyde in the feed.

As outlined above, the heat energy required for the (endothermic) reaction of the mixture of ethanol and acetaldehyde, to give 1,3-butadiene, is supplied to the adiabatic reaction zone only by the feed supplied to the adiabatic reaction zone. The temperature drop depends on conversion and insulation of the reactor: heat losses. Typically, the progress of the endothermic conversion of the mixture of ethanol and acetaldehyde to 1,3-butadiene causes a temperature drop of about 30 to 100° C. along the length of the adiabatic reaction zone depending on conversion and reaction conditions. In order to maintain high efficiency, the feed must be preheated and act as a heat carrier to supply the necessary energy to the adiabatic reaction zone for optimal conversion of ethanol and acetaldehyde to 1,3-butadiene.

Thus, according to a preferred embodiment of the process according to the invention, the temperature of the feed before contacting the supported catalyst is in the range of from 320 to 430° C., more preferably from 350 to 410° C., most preferably from 380 to 390° C.

In the process according to the invention, the adiabatic reaction zone comprising a supported catalyst and producing 1,3-butadiene is preferably operated at a pressure of from 0 to 10 barg, more preferably from 1 to 5 barg, most preferably from 1 to 3 barg.

Preferably, too high a temperature drop along the adiabatic reaction zone is avoided.

Further details regarding the stage a) reacting of a feed comprising ethanol and acetaldehyde in a reactor having at least one adiabatic reaction zone are set out in the application entitled “Adiabatically conducted process for the production of 1,3-butadiene from mixtures of ethanol and acetaldehyde” (PCT application no. PCT/EP2022/058731, attorney reference SH 1655-02WO, filed on even date herewith), the disclosure of which application is incorporated herein in its entirety. Said application entitled “Adiabatically conducted process for the production of 1,3-butadiene from mixtures of ethanol and acetaldehyde” claims priority from European patent application EP21461531.2 filed 1 Apr. 2021, which is also the filing date of European patent application EP21461530.4 (from which the present application claims priority).

Regeneration Stage b)—Stripping Step i.

Preferably, regeneration stage b) is carried out for a time period in a range of from ⅙ to ½ of the time period for which reaction stage a) is carried out. Regeneration stage b) may e.g. be carried out for a time period in a range of from ¼ to ⅓ of the duration of reaction stage a).

In accordance with the present invention, a first step of the regeneration stage b) is performed by i. stripping, carried out at a temperature in a range of from 300 to 400° C., by contacting the supported catalyst with a gas flow comprising inert gas, the gas flow having an oxygen content of 200 vol.-ppm or less. Stripping step i. desorbs and removes carbonaceous residues having a relatively low boiling point from the supported catalyst.

In stripping step i., the spent supported catalyst may additionally be washed with a solvent, in case the supported catalyst is contaminated with a high amount of carbonaceous additive. However, washing the spent supported catalyst requires a substantial decrease of the supported catalyst's temperature, and this may be detrimental to catalytic activity.

In a further preferred embodiment of the invention, total gas hourly space velocity (GHSV) in stripping step i. is in a range of from 20 to 500 h⁻¹, preferably in a range of from 50 to 300 h⁻¹. Furthermore, it is preferred that the gas flow comprising inert gas to stripping step i. is heated up to 380 to 400° C., at which temperature it contacts the supported catalyst. Because the supported catalyst to be regenerated is in an adiabatic zone, the temperature of the supported catalyst along the zone in stripping step i. falls by about 50° C., depending on heat losses, and the heat energy required for the (endothermic) removal of deposits from the spent supported catalyst in step i. is provided by the heated gas flow.

Preferably, stripping step i. requires a total time of less than 10 h, preferably less than 8 h, such as in a range of from 2 to 7 h, e.g. about 5 h.

It is preferred in accordance with the present invention that the gas flow in stripping step i. has an oxygen content of 150 vol.-ppm or less, in particular 100 vol.-ppm or less. Such low oxygen contents are typically provided when using commercial (low oxygen) nitrogen as inert gas.

Preferably, all materials provided to the supported catalyst in stripping step i. as gas flow are first mixed and then heated, and are then provided to the reaction zone comprising the supported catalyst, i.e. a gas flow comprising all inert gases, and optionally steam, is heated, and then contacts the supported catalyst.

In a preferred embodiment of the invention, the gas flow to stripping step i., is heated to a temperature of up to 400° C. (measured before contacting the supported catalyst).

Preferably, the total time period of regeneration stage b) is less than 80 h, preferably less than 70 h, more preferably less than 60 h, in particular less than 50 h.

Preferably, in regeneration stage b), the gas flow in stripping step i. comprises steam. The use of steam is advantageous because it moderates any thermal effects as may be occurring along the zone of supported catalyst. However, particular care must be taken that the temperature in step i. does not exceed 400° C. when using steam as part of the gas flow in step i.

In a preferred embodiment, and when steam is part of the gas flow to stripping step i., the gas flow (including steam) is heated to a temperature of up to 400° C. (measured before contacting the supported catalyst).

Any steam partial pressure at temperatures above 400° C., especially a high steam partial pressure, may cause undesired support sintering, by forming mobile surface hydroxyl groups that are volatilized at higher temperatures. For instance, it is known that, in Fluid Catalytic Cracking at a high-temperature of 650 to 760° C., and at a pressure of 3 atm, regeneration in steam/air mixtures leads to dealumination and destruction of zeolite crystallinity, and a loss of surface area/pore volume occurs. For these reasons, and whilst steam may be present in the gas flow in stripping step i. and first combustion step ii. according to the present invention, heated up to 400° C. only, the gas flow to second combustion step iii., which is heated up to 550° C., contains no steam.

Preferably, the gas flow in stripping step i. comprises nitrogen and steam in a ratio (vol./vol.) in a range of from 10:1 to 1:5. It is more preferred that the gas flow in stripping step i. comprises nitrogen and steam in a ratio (vol./vol.) in a range of from 5:1 to 1:2, such as in a ratio (vol./vol.) in a range of from 3:1 to 1:1. In particular, the gas flow in stripping step i. consists of nitrogen and steam in a ratio (vol./vol.) in a range of about 2:1.

Preferably, in regeneration stage b), the heated gas flow, at the end of stripping step i., contacts the supported catalyst at a temperature of 400° C. or less. More preferably, the heated gas flow, at the end of stripping step i., contacts the supported catalyst at a temperature in a range of from 380 to 400° C. It is in particular preferred that the heated gas flow, at the end of stripping step i., contacts the supported catalyst at a temperature of about 390° C.

Regeneration Stage b)—First Combustion Step ii.

Subsequent to stripping step i., the flow of inert gas is gradually replaced by a flow of gas comprising oxygen, ensuring that the oxygen content in the gas flow to first combustion step ii. does not exceed 8 vol. %. First combustion step ii. of the regeneration stage b) of the process according to the invention results in the initial oxidative removal of carbonaceous contaminants from the supported catalyst. First combustion step ii. is carried out at a temperature in a range of from 350 to 400° C. Preferably, first combustion step ii. is carried out in a gas flow comprising inert gas and oxygen, in the presence of steam, as explained above in relation to stripping step i.

Preferably, the gas flow in first combustion step ii. comprises nitrogen and steam in a ratio (vol./vol.) in a range of from 10:1 to 1:5. It is more preferred that the gas flow in first combustion step ii. comprises nitrogen and steam in a ratio (vol./vol.) in a range of from 5:1 to 1:2, such as in a ratio (vol./vol.) in a range of from 3:1 to 1:1. In particular, the gas flow in first combustion step ii. consists of nitrogen and steam in a ratio (vol./vol.) in a range of about 2:1.

Preferably, the gas flow in first combustion step ii. initially comprises steam and at the end of first combustion step ii. contains no steam. The use of steam is advantageous in step ii. because it moderates any thermal effects as may be occurring along the zone of supported catalyst due to (exothermic) oxidation of (carbonaceous) deposits.

In all embodiments of the invention, the gas used for incorporating oxygen into the gas flow of those regeneration steps that include the feeding of oxygen (namely first combustion step ii., second combustion step iii., or both first combustion step ii. and second combustion step iii.) is conveniently chosen to be air. Air has the advantage that it comprises both an inert gas and oxygen, and that the oxygen can conveniently be dosed to the gas flow, as required in order to supply the desired amount of oxygen to the gas flows comprising oxygen, namely those in regeneration steps i. and ii.

Preferably, all materials provided to the supported catalyst in first combustion step ii. as gas flow are first mixed and then heated, and are then provided to the reaction zone comprising the supported catalyst, i.e. a gas flow comprising all inert gases and all gases contributing oxygen to the gas flow (e.g. air), and optionally steam, is heated, and then contacts the supported catalyst.

More preferably, the gas flow to first combustion step ii. is heated to 380 to 400° C. before contacting the supported catalyst.

The gas flow is set such that total GHSV is kept in a range of from 50 to 500 h⁻¹, preferably in a range of from 100 to 300 h⁻¹. The total time that regeneration is performed in step ii. is preferably 40 h or less, more preferably 30 h or less, in particular, 25 h or less. During regeneration in step ii., and as a result of oxidation of carbonaceous deposits, a wave of temperature maxima moves along the zone comprising the supported catalyst. The temperature along the zone comprising the supported catalyst varies in a range of from 350 to 400° C., depending on the oxygen concentration in the gas flow to first combustion step ii.

Preferably, the gas flow to first combustion step ii. has an oxygen content in a range of from 0.75 to 7 vol. %, more preferably, the gas flow has an oxygen content in a range of from 1 to 6 vol. %.

Also, it is preferred that the oxygen content in the gas flow increases during the course of first combustion step ii. and is increased to at least 3 vol. %, more preferably is increased to at least 4 vol. %, most preferably is increased to at least 5 vol. %.

Preferably, the gas flow in first combustion step ii. initially contains less than 1 vol. % oxygen and at the end of first combustion step ii. contains oxygen in an amount in a range of from 1 to 6 vol. %.

Towards the end of first combustion step ii., and before starting second combustion step iii., the flow comprising oxygen (e.g., air) and, when present, steam, is either gradually exchanged with nitrogen in a manner that keeps the oxygen content of the gas flow to first combustion step ii. at 8 vol. % or less; or steam supply, when present in the gas flow to first combustion step ii., is stopped, keeping the gas flow to first combustion step ii. at an oxygen content of 8 vol. % or less. It is preferred that, in case steam is present in the gas flow to first combustion step ii., the amount of steam in the gas flow to first combustion step ii. is gradually reduced.

Preferably, all materials provided to the supported catalyst in second combustion step iii. as gas flow are first mixed and then heated, and are then provided to the reaction zone comprising the supported catalyst, i.e. a gas flow comprising all inert gases and all gases contributing oxygen to the gas flow (e.g. air) is heated, and then contacts the supported catalyst.

Preferably, in regeneration stage b), the heated gas flow, at the end of first combustion step ii., contacts the supported catalyst at a temperature of 400° C. or less. More preferably, the heated gas flow, at the end of first combustion step ii., contacts the supported catalyst at a temperature in a range of from 380 to 400° C. In particular, the heated gas flow, at the end of first combustion step ii., contacts the supported catalyst at a temperature of about 390° C.

Regeneration Stage b)—Second Combustion Step iii.

In the third regeneration step iii., the second combustion step, carbonaceous deposits on the supported catalyst are combusted, at a temperature of up to 550° C. Second combustion step iii. is typically carried out until the presence of carbon dioxide can no longer be detected in the effluent or, if small amounts of carbon dioxide are detected in the effluent, such carbon dioxide content no longer decreases with time. Care is taken to not exceed a maximum temperature of 550° C. in the zone of supported catalyst.

Second combustion step iii. is carried out in a gas flow comprising inert gas. As mentioned above, whereas steam can be present in the gas flow to regeneration steps i. and ii., no such addition of steam is made to the gas flow to second combustion step iii., because step iii. is carried out at a temperature exceeding 400° C.

Preferably, the gas flow to second combustion step iii. has an oxygen content in a range of from 0.75 to 7 vol. %, more preferably, the gas flow has an oxygen content in a range of from 1 to 6 vol. %

In second combustion step iii., total GHSV should preferably be kept in a range of from 20 to 500 h⁻¹, more preferably in a range of from 50 to 300 h⁻¹. Preferably, the gas flow to second combustion step iii. is preheated and contacts the supported catalyst at a temperature in a range of from 400 to 550° C. Similar to first combustion step ii., second combustion step iii. involves exothermic reactions and, consequently, a wave of local temperature maxima moves along the zone comprising the supported catalyst. The temperature of the supported catalyst in the zone is in a range of from 400 to 550° C. and depends on the oxygen concentration in the gas flow to second combustion step iii.

Typically, second combustion step iii. is carried out for a time period in a range of from 5 to 30 h, preferably 10 to 20 h, such as about 15 h. Second combustion step iii. is completed when no temperature maxima are observed along the zone of supported catalyst (i.e., when any exothermic reaction has ceased). The temperature of the supported catalyst should not exceed 550° C. and the possibility to introduce steam into the flow gas of stripping step i., or first combustion step ii., or both, significantly reduces the risk of an exothermic run-off during the combustion steps of the regeneration stage b) of the process according to the invention (i.e. combustion steps ii. and iii.). Typically, and as a safety precaution, the reactor used in accordance with the invention allows for temporary switch-off of any gas flow comprising oxygen to first combustion step i. and second combustion step ii.

Preferably, in regeneration stage b), combustion step iii. is carried out until no local temperature maximum is observed along the length of the zone comprising the supported catalyst.

Preferably, in regeneration stage b), the heated gas flow, at the end of second combustion step iii., contacts the supported catalyst at a temperature of 550° C. or less, such as in a range of from 500 to 550° C. For instance, the heated gas flow, at the end of second combustion step iii., contacts the supported catalyst at a temperature of 520 to 550° C., in particular the heated gas flow, at the end of second combustion step iii., contacts the supported catalyst at a temperature of about 540° C.

Regeneration Stage b)—Stripping Step iv.

In stripping step iv. of regeneration stage b) of the process according to the invention, the reaction zone is cooled down to the temperature desired for a subsequent reaction stage a) In stripping step iv., GHSV is preferably kept in a range of from 20 to 400 h⁻¹, more preferably in a range of from 50 to 200 h⁻¹. The temperature of the heated gas flow is gradually reduced, to reach the desired temperature for carrying out reaction stage a). Preferably, the time period for which stripping step iv. is carried out is in a range of from 1 to 10 h, typically, 2 to 8 h, such as about 5 h. Stripping step iv. is carried out subsequent to second combustion step iii. by reducing the flow of oxygen in the gas flow to the supported catalyst.

It is preferred in accordance with the present invention that the gas flow in stripping step iv. has an oxygen content of 150 vol.-ppm or less, in particular 100 vol.-ppm or less. Such low oxygen contents are typically provided when using commercial (low oxygen) nitrogen as inert gas.

Preferably, all materials provided to the supported catalyst in stripping step iv. as gas flow are first mixed and then heated, and are then provided to the reaction zone comprising the supported catalyst, i.e. a gas flow comprising all inert gases is heated, and then contacts the supported catalyst.

Preferably, in regeneration stage b), the heated gas flow, at the end of stripping step iv., contacts the supported catalyst at a temperature of 450° C. or less. The heated gas flow at the end of stripping step iv. may contact the supported catalyst at a temperature in a range of from 350 to 400° C. In particular, the heated gas flow at the end of stripping step iv. may contact the supported catalyst at a temperature of about 380° ° C.

Process for the Production of 1,3-Butadiene from Ethanol with Catalyst Regeneration

In a second aspect, the present invention relates to a process for the production of 1,3-butadiene from ethanol with catalyst regeneration comprising
x) producing acetaldehyde from ethanol in an acetaldehyde producing reactor having a reaction zone, the reaction zone of the acetaldehyde producing reactor comprising a supported or unsupported (bulk) catalyst, and
y) producing 1,3-butadiene with catalyst regeneration according to the process of the first aspect of the invention.

Preferably, the reaction zone of the acetaldehyde producing reactor according to the second aspect of the invention is an isothermal reaction zone.

Said process for the production of 1,3-butadiene from ethanol is particularly advantageous, because the acetaldehyde required can be generated from ethanol and does not have to be purchased as a raw material for the process according to the invention.

According to a preferred embodiment of the process according to the invention, the supported or unsupported (bulk) catalyst comprises one or more of zinc, copper, silver, chromium, magnesium and nickel, in particular one or more of zinc and copper.

Preferably, the acetaldehyde producing reactor comprises a supported catalyst.

According to a preferred embodiment, the support of the supported catalyst of the acetaldehyde producing reactor is selected from the group consisting of ordered and non-ordered porous silica supports, aluminium oxide supports, aluminosilicate supports, clays, other porous oxide supports, and mixtures thereof.

Preferably, the support of the supported catalyst of the acetaldehyde producing reactor is a silica support, more preferably an ordered or non-ordered porous silica support.

Preferably, the support of the supported catalyst of the acetaldehyde producing reactor has a specific surface area (SSA) in a range of from 7 to 550 m²/g, more preferably in a range of from 190 to 350 m²/g.

Preferably, the support of the supported catalyst of the acetaldehyde producing reactor has an average pore diameter in a range of from 10 to 300 Å (determined by the method of Barrett, Joyner and Halenda).

Preferably, the support of the supported catalyst of the acetaldehyde producing reactor has a pore volume in a range of from 0.2 to 1.5 ml/g (determined by the method of Barrett, Joyner and Halenda).

More preferably, the support of the supported catalyst of the acetaldehyde producing reactor is a silica support with a specific surface area in a range of from 7 to 550 m²/g, most preferably from 190 to 350 m²/g, and an average pore diameter in a range of from 10 to 300 Å, and a pore volume in a range of from 0.2 to 1.5 ml/g.

Most preferably, the support of the supported catalyst of the acetaldehyde producing reactor is an ordered or non-ordered porous silica support with a specific surface area in a range of from 7 to 550 m²/g, most preferably from 190 to 350 m²/g, and an average pore diameter in a range of from 10 to 300 Å, and a pore volume in a range of from 0.2 to 1.5 ml/g.

The supported or unsupported (bulk) catalyst in the reaction zone of the acetaldehyde producing reactor may be any (commercial) catalyst that is able to catalyse the dehydrogenation of ethanol to acetaldehyde.

Plant for the Production of 1,3-butadiene from Ethanol and Acetaldehyde

In a third aspect, the present invention relates to a plant for the production of 1,3-butadiene comprising at least one reactor for producing 1,3-butadiene from ethanol and acetaldehyde, the reactor for producing 1,3-butadiene having
a) at least one zone for producing 1,3-butadiene, the zone comprising a supported catalyst for producing 1,3-butadiene, and
b) means for feeding a feed comprising ethanol and acetaldehyde into the reactor for producing 1,3-butadiene,
the reactor for producing 1,3-butadiene having reactant heating means for heating the feed comprising ethanol and acetaldehyde before contacting the supported catalyst, the reactant heating means being sufficient to react ethanol and acetaldehyde under adiabatic conditions,
the reactor for producing 1,3-butadiene further having
c) means for regenerating the supported catalyst for producing 1,3-butadiene, comprising
   x) means for feeding a flow comprising inert gas into the reactor for producing 1,3-butadiene, and
   y) means for feeding a flow comprising oxygen into the reactor for producing 1,3-butadiene,
      the reactor for producing 1,3-butadiene having regenerant heating means for heating a flow comprising the inert gas and the oxygen before contacting the supported catalyst, the regenerant heating means being sufficient to regenerate the supported catalyst under adiabatic conditions.

Shell-and-tube heat exchangers are the preferred type of heating means to heat the various feeds (or mixture of feeds). Organic components of feed and product streams (such as ethanol and acetaldehyde) are flammable, and can be explosive within specific concentration limits when mixed with oxygen-containing gas. Therefore, for safety reasons, separate heat exchangers are used to heat gasses and gas mixtures used in the regeneration.

Preferably, the means for regenerating the supported catalyst further comprise z) means for feeding a flow comprising steam into the reactor.

Plant for the Production of 1,3-butadiene from Ethanol

In a fourth aspect, the invention relates to a plant for the production of 1,3-butadiene from ethanol, having
i. at least one reactor for producing acetaldehyde from ethanol, the reactor for producing acetaldehyde from ethanol having
a) at least one zone for producing acetaldehyde from ethanol, the zone for producing acetaldehyde comprising a supported or unsupported (bulk) catalyst for producing acetaldehyde, and
b) means for feeding a feed comprising ethanol into the reactor for producing acetaldehyde; and
ii. at least one reactor for producing 1,3-butadiene from ethanol and acetaldehyde having
a) at least one zone for producing 1,3-butadiene, the zone comprising a supported catalyst for producing 1,3-butadiene, and
b) means for feeding a feed comprising ethanol and acetaldehyde into the reactor for producing 1,3-butadiene,
the reactor for producing 1,3-butadiene having reactant heating means for heating the feed comprising ethanol and acetaldehyde before contacting the supported catalyst, the reactant heating means being sufficient to react ethanol and acetaldehyde under adiabatic conditions,
the reactor for producing 1,3-butadiene further having
c) means for regenerating the supported catalyst for producing 1,3-butadiene, comprising
   x) means for feeding a flow comprising inert gas into the reactor for producing 1,3-butadiene, and
   y) means for feeding a flow comprising oxygen into the reactor for producing 1,3-butadiene,
the reactor for producing 1,3-butadiene having regenerant heating means for heating a flow comprising the inert gas and the oxygen before contacting the supported catalyst, the regenerant heating means being sufficient to regenerate the supported catalyst under adiabatic conditions.

1. Process for the production of 1,3-butadiene from ethanol and acetaldehyde with catalyst regeneration, the process comprising
a) reacting a feed comprising ethanol and acetaldehyde in a reactor having at least one adiabatic reaction zone, the adiabatic reaction zone comprising a supported catalyst and producing 1,3-butadiene, whereby spent supported catalyst is formed;
b) regenerating the spent supported catalyst in a regeneration stage comprising the following subsequent steps:
i. a stripping step, carried out at a temperature in a range of from 300 to 400° C., by contacting the supported catalyst with a gas flow comprising inert gas, the gas flow having an oxygen content of 200 vol.-ppm or less;
ii. a first combustion step carried out at a temperature in a range of from 350 to 400° C., by contacting the supported catalyst with a gas flow comprising inert gas, the gas flow having an oxygen content in a range of from 0.2 to 8 vol. %;
iii. a second combustion step carried out at a temperature in a range of from 400 to 550° C., by contacting the supported catalyst with a gas flow comprising inert gas, the gas flow having an oxygen content in a range of from 0.2 to 8 vol. %;
iv. a stripping step carried out at a temperature in a range of from 550° C. to 300° C., by contacting the supported catalyst with a gas flow comprising inert gas, the gas flow having an oxygen content of less than 200 vol.-ppm;
wherein the gas flows to each of regeneration steps b)i. to b)iv. are first heated and then contact the supported catalyst.

2. The process according to claim 1, wherein the supported catalyst comprises one or more of tantalum, zirconium, niobium, hafnium, titanium, and tin,
preferably wherein the supported catalyst comprises tantalum,
more preferably wherein the supported catalyst comprises tantalum in an amount of from 0.1 to 10 wt %, preferably from 0.5 to 5 wt %, more preferably from 2 to 3 wt %, calculated as Ta₂O₅ and based on the total weight of the supported catalyst.

3. The process according to claim 1 or claim 2, wherein, in reaction stage a), the 1,3-butadiene producing reactor includes a first adiabatic reaction zone comprising a supported catalyst and producing 1,3-butadiene and a second adiabatic reaction zone comprising a supported catalyst and producing 1,3-butadiene,
preferably wherein the first adiabatic reaction zone comprising a supported catalyst and producing 1,3-butadiene and the second adiabatic reaction zone comprising a supported catalyst and producing 1,3-butadiene are separated by a non-reaction zone,
more preferably wherein the non-reaction zone is heated,
in particular wherein the heated non-reaction zone comprises an inert packing.

4. The process according to claim 3, wherein, in reaction stage a), an additional feed comprising acetaldehyde (and optionally ethanol) is fed into the reactor after the first adiabatic reaction zone comprising a supported catalyst and producing 1,3-butadiene,
preferably wherein the additional feed is mixed with the effluent from the first adiabatic reaction zone comprising a supported catalyst and producing 1,3-butadiene and is then fed to the second adiabatic reaction zone comprising a supported catalyst and producing 1,3-butadiene.

5. The process according to claim 4, wherein, in reaction stage a), the additional feed further comprises ethanol, and the molar ratio of ethanol to acetaldehyde in the additional feed is in the range of from 0.1 to 5, preferably 1 to 2, more preferably 1.4 to 1.8.

6. The process according to any of the preceding claims, wherein, in reaction stage a), a first 1,3-butadiene producing reactor having at least a first adiabatic reaction zone comprising a supported catalyst and producing 1,3-butadiene, and a second 1,3-butadiene producing reactor having at least a second adiabatic reaction zone comprising a supported catalyst and producing 1,3-butadiene are connected in series, and at least part of the effluent (and preferably the entire effluent) from the first 1,3-butadiene producing reactor is fed to the second 1,3-butadiene producing reactor,
more preferably wherein an additional feed comprising acetaldehyde (and optionally ethanol) is fed into the second reactor.

7. The process according any of the preceding claims, wherein regeneration stage b) is carried out for a time period in a range of from ⅙ to ½ of the time period for which reaction stage a) is carried out,
preferably wherein regeneration stage b) is carried out for a time period in a range of from ¼ to ⅓ of the duration of reaction stage a).

8. The process according to any of the preceding claims, wherein the total time period of regeneration stage b) is less than 80 h, preferably less than 70 h, more preferably less than 60 h, in particular less than 50 h.

9. The process according to any of the preceding claims, wherein, in regeneration stage b), the gas flow in stripping step i. comprises steam,
preferably wherein the gas flow in stripping step i. comprises nitrogen and steam in a ratio (vol./vol.) in a range of from 10:1 to 1:5,
more preferably wherein the gas flow in stripping step i. comprises nitrogen and steam in a ratio (vol./vol.) in a range of from 5:1 to 1:2,
most preferably wherein the gas flow in stripping step i. comprises nitrogen and steam in a ratio (vol./vol.) in a range of from 3:1 to 1:1,
in particular wherein the gas flow in stripping step i. consists of nitrogen and steam in a ratio (vol./vol.) in a range of about 2:1.

10. The process according to claim 9, wherein the gas flow in first combustion step ii. initially comprises steam and at the end of first combustion step ii. contains no steam.

11. The process according to claim 9 or claim 10, wherein the gas flow in first combustion step ii. initially contains less than 1 vol. % oxygen and at the end of first combustion step ii. contains oxygen in an amount in a range of from 1 to 6 vol. %.

12. The process according to any of the preceding claims, wherein, in regeneration stage b), combustion step iii. is carried out until no local temperature maximum is observed along the length of the zone comprising the supported catalyst.

13. The process according to any of the preceding claims, wherein, in regeneration stage b), the heated gas flow, at the end of stripping step i., contacts the supported catalyst at a temperature of 400° C. or less,
preferably the heated gas flow, at the end of stripping step i., contacts the supported catalyst at a temperature in a range of from 380 to 400° C.,
more preferably the heated gas flow, at the end of stripping step i., contacts the supported catalyst at a temperature of about 390° C.

14. The process according to any of the preceding claims, wherein, in regeneration stage b), the heated gas flow, at the end of first combustion step ii., contacts the supported catalyst at a temperature of 400° C. or less,
preferably the heated gas flow, at the end of first combustion step ii., contacts the supported catalyst at a temperature in a range of from 380 to 400° C.,
more preferably the heated gas flow, at the end of first combustion step ii., contacts the supported catalyst at a temperature of about 390° C.

15. The process according to any of the preceding claims, wherein, in regeneration stage b), the heated gas flow, at the end of second combustion step iii., contacts the supported catalyst at a temperature of 550° C. or less,
preferably the heated gas flow, at the end of second combustion step iii., contacts the supported catalyst at a temperature in a range of from 500 to 550° C.,
more preferably the heated gas flow, at the end of second combustion step iii., contacts the supported catalyst at a temperature of 520 to 550° C.,
in particular the heated gas flow, at the end of second combustion step iii., contacts the supported catalyst at a temperature of about 540° C.

16. The process according to any of the preceding claims, wherein, in regeneration stage b), the heated gas flow, at the end of stripping step iv., contacts the supported catalyst at a temperature of 450° ° C. or less,
preferably the heated gas flow, at the end of stripping step iv., contacts the supported catalyst at a temperature in a range of from 350 to 400° C.,
more preferably the heated gas flow, at the end of stripping step iv., contacts the supported catalyst at a temperature of about 380° C.

17. Process for the production of 1,3-butadiene from ethanol with catalyst regeneration comprising
x) producing acetaldehyde from ethanol in an acetaldehyde producing reactor having a reaction zone, the reaction zone of the acetaldehyde producing reactor comprising a supported or unsupported (bulk) catalyst, and
y) producing 1,3-butadiene with catalyst regeneration according to the process of any of the preceding claims,
preferably wherein the reaction zone of the acetaldehyde producing reactor is an isothermal reaction zone.

18. A plant for the production of 1,3-butadiene comprising at least one reactor for producing 1,3-butadiene from ethanol and acetaldehyde, the reactor for producing 1,3-butadiene having
a) at least one zone for producing 1,3-butadiene, the zone comprising a supported catalyst for producing 1,3-butadiene, and
b) means for feeding a feed comprising ethanol and acetaldehyde into the reactor for producing 1,3-butadiene,
the reactor for producing 1,3-butadiene having reactant heating means for heating the feed comprising ethanol and acetaldehyde before contacting the supported catalyst for producing 1,3-butadiene, the reactant heating means being sufficient to react ethanol and acetaldehyde under adiabatic conditions,
the reactor for producing 1,3-butadiene further having
c) means for regenerating the supported catalyst for producing 1,3-butadiene, comprising
x) means for feeding a flow comprising inert gas into the reactor for producing 1,3-butadiene, and
y) means for feeding a flow comprising oxygen into the reactor for producing 1,3-butadiene,
the reactor for producing 1,3-butadiene having regenerant heating means for heating a flow comprising the inert gas and the oxygen before contacting the supported catalyst for producing 1,3-butadiene, the regenerant heating means being sufficient to regenerate the supported catalyst under adiabatic conditions.

19. The plant according to claim 18, wherein the means for regenerating the supported catalyst for producing 1,3-butadiene further comprise z) means for feeding a flow comprising steam into the reactor for producing 1,3-butadiene.

20. A plant for the production of 1,3-butadiene from ethanol, having
i. at least one reactor for producing acetaldehyde from ethanol, the reactor for producing acetaldehyde from ethanol having
a) at least one zone for producing acetaldehyde from ethanol, the zone for producing acetaldehyde comprising a supported or unsupported (bulk) catalyst for producing acetaldehyde, and
b) means for feeding a feed comprising ethanol into the reactor for producing acetaldehyde; and
ii. at least one reactor for producing 1,3-butadiene from ethanol and acetaldehyde, the reactor for producing 1,3-butadiene having
a) at least one zone for producing 1,3-butadiene, the zone comprising a supported catalyst for producing 1,3-butadiene, and
b) means for feeding a feed comprising ethanol and acetaldehyde into the reactor for producing 1,3-butadiene,
the reactor for producing 1,3-butadiene having reactant heating means for heating the feed comprising ethanol and acetaldehyde before contacting the supported catalyst for producing 1,3-butadiene, the reactant heating means being sufficient to react ethanol and acetaldehyde under adiabatic conditions,
the reactor for producing 1,3-butadiene further having
c) means for regenerating the supported catalyst for producing 1,3-butadiene, comprising
x) means for feeding a flow comprising inert gas into the reactor for producing 1,3-butadiene, and
y) means for feeding a flow comprising oxygen into the reactor for producing 1,3-butadiene,
the reactor for producing 1,3-butadiene having regenerant heating means for heating a flow comprising the inert gas and the oxygen before contacting the supported catalyst for producing 1,3-butadiene, the regenerant heating means being sufficient to regenerate the supported catalyst under adiabatic conditions.
